(19) United States
(12) Patent Application Publication
(10) Pub. No.: US 2026/0294398 A1
(43) Pub. Date: Oct. 1, 2026

(54) ULTRASOUND SYSTEM AND CONTROL METHOD OF ULTRASOUND SYSTEM

(71) Applicant: FUJIFILM Corporation, Tokyo (JP)

(72) Inventors: Hideki YAMAGISHI, Kanagawa (JP); Hiroyuki KARASAWA, Kanagawa (JP); Koji SHIMOMURA, Kanagawa (JP)

(73) Assignee: FUJIFILM Corporation, Tokyo (JP)

(21) Appl. No.: 19/562,810

(22) Filed: Mar. 11, 2026

(30) Foreign Application Priority Data
Mar. 27, 2025 (JP) 2025-053500
Sep. 25, 2025 (JP) 2025-158973

(51) Int. Cl. A61B 8/00

(52) U.S. Cl. CPC A61B 8/4472; A61B 8/5207; A61B 8/56; A61B 2560/0242

(57) ABSTRACT

Provided are an ultrasound system and a control method of an ultrasound system capable of securing a sufficient remaining level of a built-in battery of an ultrasound probe while preventing a surface temperature of the ultrasound probe from exceeding an upper limit temperature.

The ultrasound system includes a processor configured to charge the built-in battery of the ultrasound probe with charging power set according to operation information of the ultrasound probe by using power supplied from an outside of the ultrasound probe.

### CROSS-REFERENCE TO RELATED APPLICATIONS

The present application claims priority under 35 U.S.C. § 119 to Japanese Patent Application No. 2025-053500, filed on Mar. 27, 2025, and Japanese Patent Application No. 2025-158973, filed on Sep. 25, 2025. Each of the above applications is hereby expressly incorporated by reference, in its entirety, into the present application.

### BACKGROUND OF THE INVENTION

### 1. Field of the Invention

The present invention relates to an ultrasound system including an ultrasound probe that incorporates a battery and a control method of the ultrasound system.

### 2. Description of the Related Art

In the related art, an ultrasound examination has been performed in which an ultrasound image representing a tomographic plane inside a subject is captured using a so-called ultrasound probe, and the inside of the subject is examined based on the captured ultrasound image. In order to smoothly perform such an ultrasound examination, for example, as disclosed in JP2013-135557A, an ultrasound probe that incorporates a battery has been developed.

### SUMMARY OF THE INVENTION

In general, in safety standards such as IEC 60601-2-37, which is an international standard related to individual requirements for basic safety and basic performance of a medical ultrasound diagnostic apparatus and a monitor device, it is defined that a surface temperature of the ultrasound probe does not exceed an upper limit temperature such as 43° C. in order to prevent a burn of a subject and a user. The ultrasound probe may generate heat due to charging the built-in battery, and the built-in battery may not be charged in a case where the temperature of the ultrasound probe may exceed the predetermined upper limit temperature by the safety standard, for example, in a case where the built-in battery is charged in a state where the power supply of the ultrasound probe is turned on immediately after the ultrasound probe is used. In addition, in a case where the number of times of charging the built-in battery cannot be secured due to such a situation, there is a concern that the remaining level of the built-in battery may be insufficient during the ultrasound examination.

The present invention has been made to solve such a problem in the related art, and an object of the present invention is to provide an ultrasound system and a control method of the ultrasound system capable of securing a sufficient remaining level of a built-in battery of an ultrasound probe while preventing a surface temperature of the ultrasound probe from exceeding an upper limit temperature.

The above object can be achieved with the following configurations.
[1] An ultrasound system in which an ultrasound probe including a transducer array and a built-in battery is wirelessly connected to an ultrasound apparatus main body including a processor,
in which the processor is configured to charge the built-in battery of the ultrasound probe with charging power set according to operation information of the ultrasound probe by using power supplied from an outside of the ultrasound probe.
[2] The ultrasound system according to [1], in which the operation information is information representing whether a power supply of the ultrasound probe is in an on state or an off state, and
the processor is configured to charge the built-in battery with charging power different depending on whether the power supply of the ultrasound probe is in the on state or the off state.
[3] The ultrasound system according to [2], in which the processor is configured to charge the built-in battery with predetermined first charging power in the off state of the power supply, and to charge the built-in battery with charging power smaller than the first charging power in the on state of the power supply.
[4] The ultrasound system according to [2] or [3], in which the processor is configured to charge the built-in battery with charging power different depending on whether the ultrasound probe is in a connection state of being wirelessly connected to the ultrasound apparatus main body or in a non-connection state of being not wirelessly connected to the ultrasound apparatus main body.
[5] The ultrasound system according to [4], in which the processor is configured to charge the built-in battery with predetermined second charging power in the non-connection state, and to charge the built-in battery with third charging power smaller than the second charging power in the connection state.
[6] The ultrasound system according to [1], further including a probe temperature sensor that detects a temperature of the ultrasound probe,
in which the operation information is information representing the temperature detected by the probe temperature sensor, and
the processor is configured to control the charging power of the built-in battery based on the temperature.
[7] The ultrasound system according to any one of [2] to [5], further including a probe temperature sensor that detects a temperature of the ultrasound probe,
in which the processor is configured to control the charging power of the built-in battery by taking into account the temperature of the ultrasound probe detected by the probe temperature sensor.
[8] The ultrasound system according to [1], further including:
a charger that supplies power to the ultrasound probe; and
a charger temperature sensor that detects a temperature of the charger,
in which the processor is configured to use information representing the temperature of the charger detected by the charger temperature sensor as the operation information,
to control the charging power of the built-in battery based on the temperature of the charger detected by the charger temperature sensor, and
to charge the built-in battery with the controlled charging power.
[9] The ultrasound system according to any one of [2] to [5], further including:
a charger that supplies power to the ultrasound probe; and
a charger temperature sensor that detects a temperature of the charger,
in which the processor is configured to control the charging power of the built-in battery by taking into account the temperature of the charger detected by the charger temperature sensor, and
to charge the built-in battery with the controlled charging power by using the power supplied from the charger.
[10] The ultrasound system according to [6] or [7], further including a cooling device that cools the ultrasound probe,
in which the processor is configured to cool the ultrasound probe by using the cooling device according to the temperature of the ultrasound probe detected by the probe temperature sensor.
[11] The ultrasound system according to [8] or [9], further including a cooling device that cools the ultrasound probe,
in which the processor is configured to cool the ultrasound probe by using the cooling device according to the temperature of the charger detected by the charger temperature sensor.
[12] The ultrasound system according to any one of [1] to [11], in which the ultrasound probe includes a power receive coil, and
the processor is configured to charge the built-in battery by using the power wirelessly supplied from the outside of the ultrasound probe via the power receive coil.
[13] The ultrasound system according to any one of [1] to [12], in which the processor is configured to stop transmission and reception of ultrasound waves in the transducer array in a state where the built-in battery is being charged.
[14] The ultrasound system according to any one of [1] to [13], in which the processor is configured to maintain wireless connection between the ultrasound probe and the ultrasound apparatus main body in a state where the built-in battery is being charged.
[15] The ultrasound system according to any one of [1] to [14], in which the ultrasound probe includes a wireless communication circuit that performs wireless communication with the ultrasound apparatus main body, and
the processor is configured to control the charging power of the built-in battery by taking into account whether the wireless communication circuit is in an on state or an off state.
[16] The ultrasound system according to any one of [1] to [15], in which the ultrasound probe includes a transmission and reception circuit that performs transmission and reception of ultrasound waves using the transducer array, and
the processor is configured to control the charging power of the built-in battery by taking into account whether the transmission and reception circuit is in an on state or an off state.
[17] The ultrasound system according to any one of [1] to [16], in which the processor is configured to control the charging power of the built-in battery by taking into account a remaining level of the built-in battery.
[18] The ultrasound system according to any one of [1] to [17], in which the processor is configured to control the charging power of the built-in battery by taking into account a plan to perform an ultrasound examination.
[19] The ultrasound system according to any one of [1] to [18], in which the processor is configured to control the charging power of the built-in battery by taking into account an operation time of a facility that performs an ultrasound examination.
[20] The ultrasound system according to any one of [1] to [19], in which the processor is configured to control the charging power of the built-in battery by taking into account a date and time set by a user.
[21] The ultrasound system according to any one of [1] to [20], in which the processor is configured to control the charging power of the built-in battery by taking into account operation information of the ultrasound apparatus main body.
[22] A control method of an ultrasound system in which an ultrasound probe including a transducer array and a built-in battery is wirelessly connected to an ultrasound apparatus main body, the control method including:
charging the built-in battery of the ultrasound probe with charging power set according to operation information of the ultrasound probe by using power supplied from an outside of the ultrasound probe.

In the present invention, the ultrasound system charges the built-in battery of the ultrasound probe with the charging power set according to the operation information of the ultrasound probe by using the power supplied from the outside of the ultrasound probe, so that the sufficient remaining level of the built-in battery of the ultrasound probe can be secured while the surface temperature of the ultrasound probe is prevented from exceeding the upper limit temperature.

### BRIEF DESCRIPTION OF THE DRAWINGS

FIG. 1 is a block diagram showing a configuration of an ultrasound system according to a first embodiment of the present invention.

FIG. 2 is a block diagram showing an internal configuration of an ultrasound probe and an ultrasound apparatus main body according to the first embodiment of the present invention.

FIG. 3 is a block diagram showing an internal configuration of a transmission and reception circuit according to the first embodiment of the present invention.

FIG. 4 is a block diagram showing an internal configuration of an image generation unit according to the first embodiment of the present invention.

FIG. 5 is a diagram showing an example of a housing of the ultrasound probe according to the first embodiment of the present invention.

FIG. 6 is a block diagram showing an internal configuration of a charger according to the first embodiment of the present invention.

FIG. 7 is a flowchart showing an operation of the ultrasound system according to the first embodiment of the present invention.

FIG. 8 is a block diagram showing an internal configuration of an ultrasound probe and an ultrasound apparatus main body according to a second embodiment of the present invention.

FIG. 9 is a block diagram showing an internal configuration of a charger according to a third embodiment of the present invention.

FIG. 10 is a block diagram showing an internal configuration of an ultrasound probe and an ultrasound apparatus main body according to a fourth embodiment of the present invention.

### DESCRIPTION OF THE PREFERRED EMBODIMENTS

Hereinafter, embodiments of the invention will be described with reference to the accompanying drawings.

The following configuration requirements are described based on a representative embodiment of the present invention, but the present invention is not limited to the embodiment.

In the present specification, a numerical range represented by “to” means a range including numerical values described before and after “to”, both ends inclusive, as a lower limit value and an upper limit value.

In the present specification, “identical” and “same” are construed to include an error range generally allowable in the technical field.

### First Embodiment

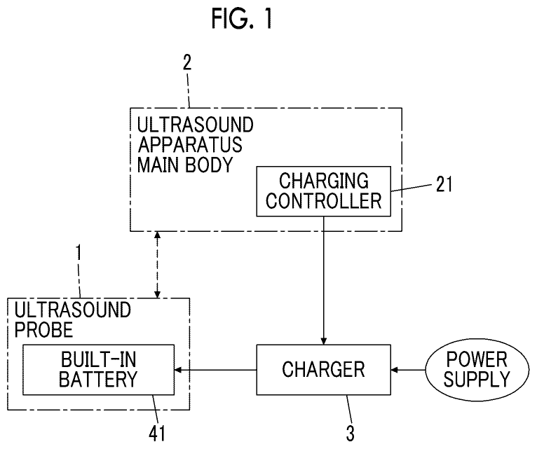

FIG. 1 shows a configuration of an ultrasound system according to a first embodiment of the present invention. The ultrasound system includes an ultrasound probe 1, an ultrasound apparatus main body 2 that is wirelessly connected to the ultrasound probe 1, and a charger 3 that is connected to the ultrasound probe 1 and the ultrasound apparatus main body 2.

The ultrasound probe 1 is wirelessly connected to the ultrasound apparatus main body 2, and transmits and receives the ultrasound waves to and from the inside of the subject to capture an ultrasound image. The ultrasound probe 1 incorporates a built-in battery 41. The built-in battery 41 is charged by being supplied with power from the charger 3. The internal configuration of the ultrasound probe 1 will be described below.

The charger 3 is connected to an alternating current (AC) power supply or the like having a specified voltage value, converts an AC voltage from the AC power supply or the like into a predetermined direct current (DC) voltage, and supplies power to the ultrasound probe 1 with the converted voltage. The charger 3 includes, for example, a so-called alternating current (AC) adapter or the like.

The ultrasound apparatus main body 2 includes a charging controller 21 that controls charging power to the built-in battery 41 of the ultrasound probe 1 by using the power supplied from the charger 3. The ultrasound apparatus main body 2 may be, for example, a so-called handheld type such as a tablet or a smartphone, or may be a so-called stationary type connected to an AC power supply or the like. Hereinafter, an example in which the ultrasound apparatus main body 2 is the handheld type will be mainly described, but the present invention is not particularly limited thereto. The internal configuration of the ultrasound apparatus main body 2 and the charging controller 21 will be described below.

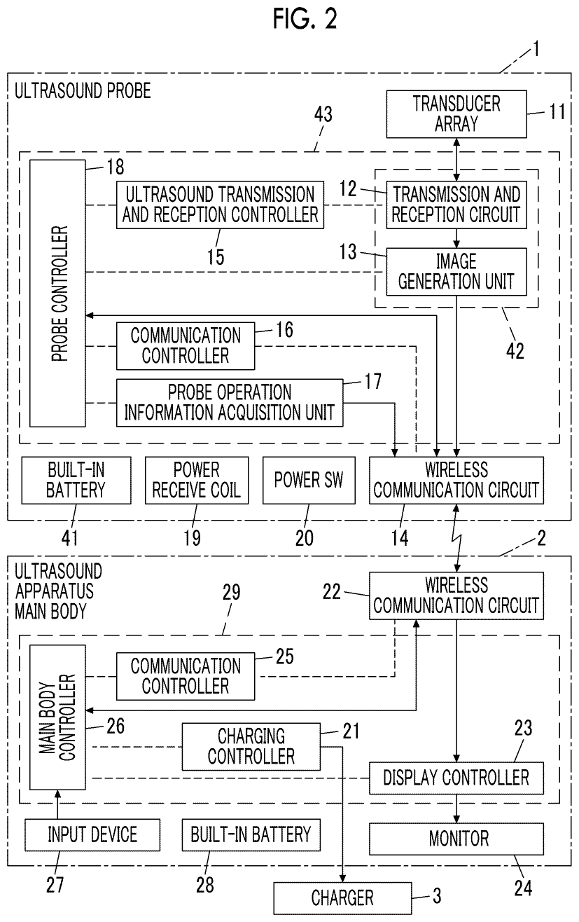

FIG. 2 shows the internal configuration of the ultrasound probe 1 and the ultrasound apparatus main body 2. The ultrasound probe 1 includes a transducer array 11, and a transmission and reception circuit 12, an image generation unit 13, and a wireless communication circuit 14 are sequentially connected to the transducer array 11. An ultrasound transmission and reception controller 15 is connected to the transmission and reception circuit 12. A communication controller 16 and a probe operation information acquisition unit 17 are connected to the wireless communication circuit 14. A probe controller 18 is connected to the image generation unit 13, the ultrasound transmission and reception controller 15, the communication controller 16, and the probe operation information acquisition unit 17. The probe controller 18 is also connected to the wireless communication circuit 14. In addition, the ultrasound probe 1 includes a power receive coil 19, a power switch (power SW) 20, and the built-in battery 41.

The transmission and reception circuit 12 and the image generation unit 13 constitute an image acquisition unit 42. A processor 43 for the ultrasound probe 1 is configured by the transmission and reception circuit 12, the image generation unit 13, the ultrasound transmission and reception controller 15, the communication controller 16, the probe operation information acquisition unit 17, and the probe controller 18.

The ultrasound apparatus main body 2 includes a wireless communication circuit 22, and a display controller 23 and a monitor 24 are sequentially connected to the wireless communication circuit 22. A communication controller 25 is connected to the wireless communication circuit 22. In addition, the ultrasound apparatus main body 2 includes the charging controller 21 that is connected to the charger 3. A main body controller 26 is connected to the charging controller 21, the wireless communication circuit 22, the display controller 23, and the communication controller 25. An input device 27 is connected to the main body controller 26. In addition, the ultrasound apparatus main body 2 includes a built-in battery 28.

A processor 29 for the ultrasound apparatus main body 2 is configured by the charging controller 21, the display controller 23, the communication controller 25, and the main body controller 26.

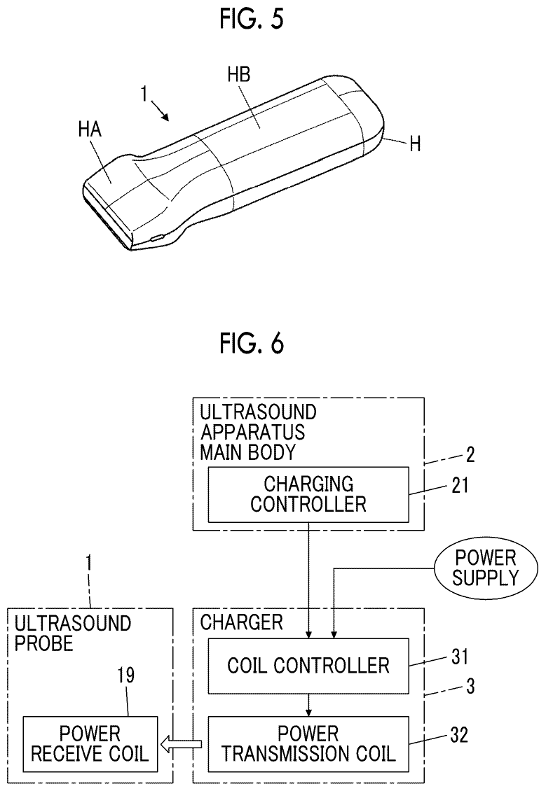

For example, as shown in FIG. 5, the ultrasound probe 1 includes a housing H that accommodates an electric circuit and the like. The housing H is formed with an array accommodation portion HA that accommodates the transducer array 11 and that comes into contact with a body surface of the subject during the ultrasound examination, and a grip portion HB for a user to grip.

The transducer array 11 of the ultrasound probe 1 has a plurality of ultrasonic transducers arranged in a one-dimensional or two-dimensional manner. In accordance with a drive signal supplied from the transmission and reception circuit 12, each of the ultrasound transducers transmits ultrasound and receives an ultrasound echo from a subject to output a signal based on the ultrasound echo. Each ultrasound oscillator is configured by forming an electrode at both ends of a piezoelectric body consisting of, for example, a piezoelectric ceramic represented by lead zirconate titanate (PZT), a polymer piezoelectric element represented by polyvinylidene difluoride (PVDF), and a piezoelectric single crystal represented by a lead magnesium niobate-lead titanate (PMN-PT).

The image acquisition unit 42 configured by the transmission and reception circuit 12 and the image generation unit 13 acquires an ultrasound image in which a tomographic plane inside the subject is captured by transmitting and receiving an ultrasound beam using the ultrasound probe 1.

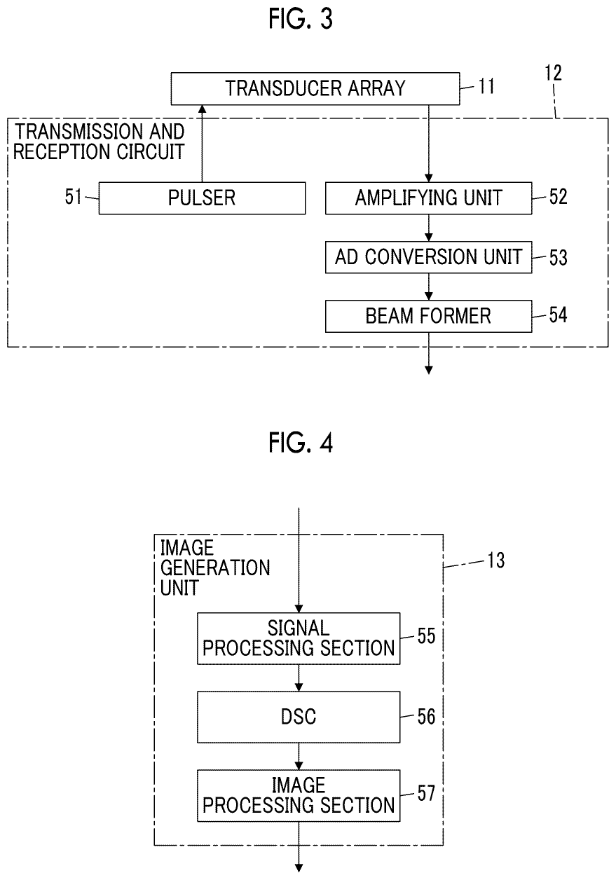

The transmission and reception circuit 12 transmits the ultrasound wave from the transducer array 11 and generates a sound ray signal based on a reception signal acquired by the transducer array 11, under the control of the probe controller 18. As shown in FIG. 3, the transmission and reception circuit 12 includes a pulser 51 connected to the transducer array 11, and an amplification section 52, an analog-to-digital (AD) conversion section 53, and a beam former 54 that are sequentially connected in series to the transducer array 11.

The pulser 51 includes, for example, a plurality of pulse generators, and adjusts an amount of delay of each of drive signals and supplies the drive signals to the plurality of ultrasound transducers such that ultrasound waves transmitted from the plurality of ultrasound transducers of the transducer array 11 form an ultrasound beam based on a transmission delay pattern selected according to a control signal from the ultrasound transmission and reception controller 15 and the probe controller 18. As described above, in a case in which a pulsed or continuous wave-like voltage is applied to the electrodes of the ultrasound transducer of the transducer array 11, the piezoelectric material expands and contracts to generate pulsed or continuous wave-like ultrasound from each of the ultrasound transducers, whereby the ultrasound beam is formed from the combined wave of the ultrasound.

The transmitted ultrasound beam is, for example, reflected by a target such as a part of the subject and propagates toward the transducer array 11 of the ultrasound probe 1. The ultrasound echo that propagates toward the transducer array 11 in this manner is received by each of the ultrasound transducers that constitute the transducer array 11. In this case, each of the ultrasound transducers that constitute the transducer array 11 receives the propagating ultrasound echo to expand and contract to generate a reception signal which is an electrical signal, thereby outputting these reception signals to the amplification section 52.

The amplification section 52 amplifies the signal input from each of the ultrasound transducers that constitute the transducer array 11 and transmits the amplified signal to the AD conversion section 53. The AD conversion section 53 converts the signal transmitted from the amplification section 52 into digital reception data. The beam former 54 performs so-called reception focus processing by applying and adding the delay to each reception data received from the AD conversion section 53. By the reception focus processing, each reception data, which is converted by the AD conversion section 53, is phase-added, and the sound ray signal in which the focus of the ultrasound echo is narrowed down is acquired.

As shown in FIG. 4, the image generation unit 13 has a configuration in which a signal processing section 55, a digital scan converter (DSC) 56, and an image processing section 57 are sequentially connected in series.

The signal processing section 55 corrects attenuation by distance of the sound ray signal received from the transmission and reception circuit 12 in accordance with depths of reflection positions of the ultrasound waves using a sound speed value set by the probe controller 18 and then performs envelope detection processing on the sound ray signal to generate a B-mode image signal that is tomographic image information related to tissues inside the subject.

The DSC 56 converts (raster-converts) the B-mode image signal generated by the signal processing section 55 into an image signal following a normal television signal scanning method.

The image processing section 57 performs various necessary image processing such as gradation processing on the B-mode image signal input from the DSC 56, and then transmits the B-mode image signal to the wireless communication circuit 14. The B-mode image signal, which is image-processed by the image processing section 57, will be referred to as an ultrasound image.

The ultrasound transmission and reception controller 15 controls the transmission and reception circuit 12 to transmit and receive the ultrasound waves in transducer array 11 under predetermined transmission and reception conditions. The predetermined transmission and reception conditions may include, for example, a transmission pattern of the ultrasound waves, a transmission focus position of the ultrasound waves, a reception focus position, a display depth of the ultrasound image, and conditions of a gain and a dynamic range of the reception signal.

The wireless communication circuit 14 is a circuit that wirelessly transmits the ultrasound image and the like acquired by the image acquisition unit 42 to the ultrasound apparatus main body 2, and wirelessly transmits and receives other necessary information to and from the ultrasound apparatus main body 2.

The communication controller 16 controls the transmission and reception of information to and from the ultrasound apparatus main body 2 by the wireless communication circuit 14. The communication controller 16 can cause the wireless communication circuit 22 to perform, for example, transmission and reception of information for the ultrasound probe 1 to be wirelessly connected to the ultrasound apparatus main body 2, transmission of the ultrasound image to the ultrasound apparatus main body 2, transmission of information representing whether a power supply of the ultrasound probe 1 is in the on state or the off state, and the like.

The power switch 20 is a switch for turning on or off the power supply of the ultrasound probe 1. The power switch 20 is operated by, for example, the user.

The probe operation information acquisition unit 17 acquires the operation information of the ultrasound probe 1 in the ultrasound probe 1. The probe operation information acquisition unit 17 can acquire, for example, information representing whether the power supply of the ultrasound probe 1 is in the on state or the off state by the power switch 20 as the operation information of the ultrasound probe 1. The operation information of the ultrasound probe 1 acquired by the probe operation information acquisition unit 17 is transmitted to the ultrasound apparatus main body 2 via the wireless communication circuit 14.

The power receive coil 19 receives the power supplied from the charger 3 by sensing a magnetic field emitted from an external wireless charger. In addition, the power receive coil 19 emits a response signal in response to a search signal transmitted from the charger 3.

The built-in battery 41 of the ultrasound probe 1 supplies power to each unit of the ultrasound probe 1. The built-in battery 41 is charged by receiving power from the charger 3 via the power receive coil 19, for example. As the built-in battery 41, various batteries can be used, but for example, a so-called lithium ion battery can be used.

The probe controller 18 controls each unit of the ultrasound probe 1 based on a control program or the like stored in advance.

The wireless communication circuit 22 of the ultrasound apparatus main body 2 is a circuit that wirelessly receives the ultrasound image and the like from the ultrasound probe 1, and wirelessly transmits and receives necessary information such as information for the ultrasound apparatus main body 2 to be wirelessly connected to the ultrasound probe 1.

The communication controller 25 controls the transmission and reception of information to and from the ultrasound probe 1 by the wireless communication circuit 22. The communication controller 25 can cause the wireless communication circuit 22 to perform, for example, reception of the ultrasound image from the ultrasound probe 1, reception of the operation information of the ultrasound probe 1, and transmission and reception of information for the ultrasound apparatus main body 2 to be wirelessly connected to the ultrasound probe 1.

The display controller 23 performs predetermined processing on the ultrasound image and the like received from the ultrasound probe 1 under the control of the main body controller 26, and displays the ultrasound image and the like on the monitor 24.

The monitor 24 is a monitor for displaying the ultrasound image and the like from the ultrasound probe 1 under the control of the display controller 23, and includes a display device such as a liquid crystal display (LCD), or an organic electroluminescence (EL) display.

By the usage status of the ultrasound probe 1, the life of the built-in battery 41 may be significantly shortened.

For example, as the voltage used to charge the built-in battery 41 of the ultrasound probe 1 is higher, the built-in battery 41 can be rapidly charged to a large capacity, but in a case where the built-in battery 41 is repeatedly charged to a charging capacity unique to the built-in battery 41, that is, repeatedly charged to full charge, by the high charging voltage, the life of the built-in battery 41 may be shortened, that is, the capacity that can be charged at once may be reduced. Examples of a situation in which the built-in battery 41 is charged a plurality of times in a short period include a situation in which a state in which the built-in battery 41 is charged by the user with the power supply of the ultrasound probe 1 in the on state during the ultrasound examination and a state in which the ultrasound probe 1 is used without charging are alternately repeated.

In addition, in a case where the power supply of the ultrasound probe 1 is in the on state, the ultrasound probe 1 may generate heat due to driving. Further, the ultrasound probe 1 may generate heat due to the rapid charging of the built-in battery 41, and in a case where the built-in battery 41 supplies a large amount of power with the power supply of the ultrasound probe 1 in the on state, the built-in battery 41 further generates heat. Therefore, for example, in a case where the built-in battery 41 is rapidly charged in a case where the power supply of the ultrasound probe 1 is in the on state, the temperature of the built-in battery 41 is likely to increase due to, for example, three elements of driving of the ultrasound probe 1, rapid charging, and a combination thereof. It is known that maintaining the built-in battery 41 at, for example, a high temperature of 40° C. or higher is one of the factors that shorten the life of the built-in battery 41.

In addition, examples of a situation in which the built-in battery 41 is stably maintained in a charged state for a sufficient time include a situation in which the ultrasound examination of the subject is not performed. In a case where the ultrasound examination of the subject is not performed, the power supply of the ultrasound probe 1 is often turned off in order to suppress power consumption of the built-in battery 41.

Therefore, the charging controller 21 controls the charging power to the built-in battery 41 such that the built-in battery 41 is charged with charging power different depending on whether the power supply of the ultrasound probe 1 is in the on state or the off state, by using the power supplied from the outside of the ultrasound probe 1, that is, the charger 3, by referring to the operation information of the ultrasound probe 1 transmitted from the ultrasound probe 1. The charging controller 21 can charge the built-in battery 41 with predetermined first charging power in the off state of the power supply of the ultrasound probe 1, and can charge the built-in battery 41 with charging power smaller than the first charging power in the on state of the power supply of the ultrasound probe 1. Here, the charging power refers to at least one of a charging voltage or a charging current.

As a result, the number of times of charging with the high charging power can be reduced while maintaining the life of the built-in battery 41 of the ultrasound probe 1, and a sufficient remaining level of the built-in battery 41 can be secured in the ultrasound examination. In addition, since the built-in battery 41 is charged with the charging power smaller than the first charging power in the on state of the power supply of the ultrasound probe 1, the influence of increase in the temperature of the built-in battery 41 on the life of the built-in battery 41 can be reduced.

In addition, the charging controller 21 can also charge the built-in battery 41 with charging power different depending on whether the ultrasound probe 1 is in a connection state of being wirelessly connected to the ultrasound apparatus main body 2 or the ultrasound probe 1 is in a non-connection state of being not wirelessly connected to the ultrasound apparatus main body 2. The charging controller 21 determines that the ultrasound probe 1 is less likely to be used in the non-connection state than in the connection state, and can, for example, charge the built-in battery 41 with predetermined second charging power in the non-connection state, and can charge the built-in battery 41 with third charging power smaller than the second charging power in the connection state. In addition, since the power consumption in the ultrasound probe 1 is larger in the connection state than in the non-connection state, the life of the built-in battery 41 can be maintained longer by further reducing the charging power. The first charging power in the off state of the power supply of the ultrasound probe 1 can be set to be larger than the second charging power in the connection state.

The input device 27 is an input device for the user to perform an input operation, and is configured by, for example, a device such as a keyboard, a mouse, a trackball, a touchpad, and a touch sensor disposed in a state of being superimposed on the monitor 24.

The built-in battery 28 of the ultrasound apparatus main body 2 supplies power to each unit of the ultrasound apparatus main body 2. The built-in battery 28 is charged by, for example, an external power supply device (not shown) connected to an AC power supply having a specified voltage value and configured by a so-called alternating current (AC) adapter or the like. As the built-in battery 28, various batteries can be used, but for example, a lithium ion battery can be used.

The main body controller 26 controls each unit of the ultrasound apparatus main body 2 based on a control program or the like stored in advance.

For example, as shown in FIG. 6, the charger 3 includes a coil controller 31 and a power transmission coil 32 connected to the coil controller 31. The coil controller 31 is connected to the charging controller 21 of the ultrasound apparatus main body 2 and an external power supply.

The coil controller 31 receives control information on the charging power from the charging controller 21 of the ultrasound apparatus main body 2 to the ultrasound probe 1, and controls the power transmitted from the power transmission coil 32 to the power receive coil 19 of the ultrasound probe 1 based on the received control information. Although not shown, the coil controller 31 constitutes a processor.

The power transmission coil 32 transmits the power to the power receive coil 19 by generating a magnetic field to the power receive coil 19 under the control of the coil controller 31.

In the present embodiment, each processing of the processor 43 of the ultrasound probe 1, the processor 29 of the ultrasound apparatus main body 2, and the processor of the charger 3 is executed by any computer. In addition, any computer may execute these processes by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the order described above and may be changed as appropriate. Any computer may be a general-purpose computer, a computer for a specific use, a workstation, or another system capable of executing each processing.

The processor 43 of the ultrasound probe 1, the processor 29 of the ultrasound apparatus main body 2, and the processor of the charger 3 may be composed of one or a plurality of hardware, and the type of the hardware is not limited. For example, the processor 43 of the ultrasound probe 1, the processor 29 of the ultrasound apparatus main body 2, and the processor of the charger 3 may be composed of a central processing unit (CPU), a micro processing unit (MPU), a programmable logic device such as a field programmable gate array (FPGA), a dedicated circuit for executing specific processing such as an application specific integrated circuit (ASIC), a graphics processing unit (GPU), a neural processing unit (NPU), or hardware. In addition, the types of hardware may be a combination of different types of hardware. In a case in which the plurality of types of hardware are configured to execute one or a plurality of types of processing of a certain processor, the plurality of types of hardware may exist in devices physically separated from each other or may exist in the same device. In addition, in any embodiment, an order of each processing by the processor 43 of the ultrasound probe 1, the processor 29 of the ultrasound apparatus main body 2, and the processor of the charger 3 is not limited to the above-described order, and may be changed as appropriate. The hardware is composed of an electric circuit (circuitry) in which circuit elements such as semiconductor elements are combined.

Furthermore, the program may be software such as firmware or a microcode. In addition, the program may be, for example, a program module group, and each function thereof may be realized by a processor configured to execute each function. The program may be a program code or a plurality of code segments stored in one or a plurality of non-transitory computer-readable media (for example, a storage medium or other storage). The program may be stored in a plurality of non-transitory computer-readable media existing in devices physically separated from each other. The program code or code segment may represent any combination of a procedure, a function, a subprogram, a routine, a subroutine, a module, a software package, a class, or an instruction, a data structure, or a program statement. The program code or code segment may be connected to another code segment or a hardware circuit by transmitting and receiving information, data, an argument, a parameter, or a content of a memory.

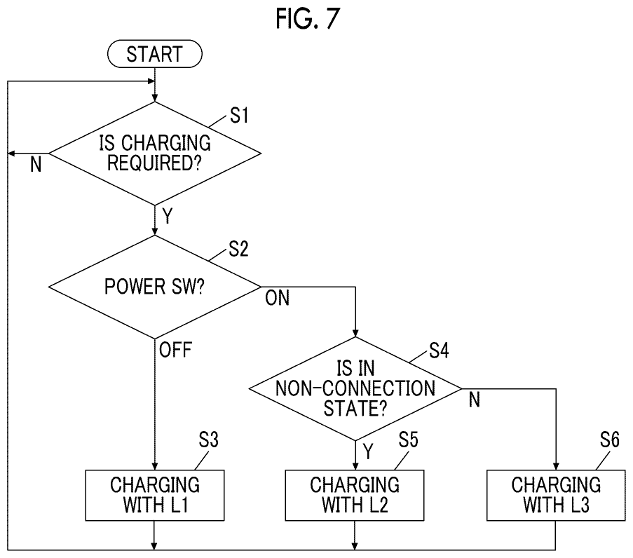

Next, an operation of charging the built-in battery 41 of the ultrasound probe 1 in the ultrasound system according to the first embodiment of the present invention will be described with reference to the flowchart of FIG. 7.

In step S1, the charging controller 21 determines whether or not charging of the built-in battery 41 of the ultrasound probe 1 is required. The charging controller 21 can determine that charging is required in a case where the built-in battery 41 is in a state in which charging is possible, for example, by detecting a connection state between the ultrasound probe 1 and the charger 3. The charging controller 21 can determine that charging is required in a case where the ultrasound probe 1 is connected to the charger 3, for example. In addition, the charging controller 21 can determine that charging is not required in a case where the ultrasound probe 1 is not connected to the charger 3.

The processing of step S1 is repeated as long as it is determined in step S1 that charging of the built-in battery 41 of the ultrasound probe 1 is not required. In a case where it is determined in step S1 that charging of the built-in battery 41 of the ultrasound probe 1 is required, the processing proceeds to step S2.

In step S2, the charging controller 21 determines whether the power supply of the ultrasound probe 1 is in the on state or the off state. In a case where it is determined in step S2 that the power supply of the ultrasound probe 1 is in the off state, the processing proceeds to step S3.

In step S3, the charging controller 21 charges the built-in battery 41 of the ultrasound probe 1 with first charging power L1. The charging with the first charging power L1 is, for example, charging with a high charging voltage such as 4.2 V, and the built-in battery 41 can be charged to the maximum capacity of the built-in battery 41. In the off state of the power supply of the ultrasound probe 1, for example, the number of times of charging is usually about once a day, such as at night when the user does not use the ultrasound probe 1, and the built-in battery 41 is stably charged for a sufficient time, so that the number of times of charging is usually small. In addition, in the off state of the power supply of the ultrasound probe 1, since the ultrasound probe 1 does not generate heat due to driving, the temperature of the built-in battery 41 is less likely to increase as compared with the on state of the power supply of the ultrasound probe 1. Therefore, with step S3, the built-in battery 41 can be charged to the maximum capacity while maintaining the life of the built-in battery 41.

In a case where it is determined in step S2 that the power supply of the ultrasound probe 1 is in the on state, the processing proceeds to step S4. In step S4, the charging controller 21 determines whether the ultrasound probe 1 is in a non-connection state with respect to the ultrasound apparatus main body 2 by referring to the operation information of the ultrasound probe 1 received from the ultrasound probe 1 via the wireless communication circuit 22. In a case where it is determined in step S4 that the ultrasound probe 1 is in the non-connection state with respect to the ultrasound apparatus main body 2, the processing proceeds to step S5.

In step S5, the charging controller 21 charges the built-in battery 41 of the ultrasound probe 1 with second charging power L2, such as a charging voltage of 4.1 V, which is smaller than the first charging power L1 in step S3. In a case where the power supply of the ultrasound probe 1 is in the on state but the ultrasound probe 1 is not connected to the ultrasound apparatus main body 2, for example, the ultrasound probe 1 is often used immediately before or after the ultrasound examination of the subject. Therefore, in a case where the ultrasound probe 1 is in the non-connection state, for example, the frequency of charging the built-in battery 41 may be high as compared with the off state of the power supply of the ultrasound probe 1, and the number of times the remaining level of the built-in battery 41 reaches the maximum capacity corresponding to the charging power used increases. In addition, in the on state of the power supply of the ultrasound probe 1, the power consumption is high as compared with the off state, and the temperature of the built-in battery 41 is likely to increase. By charging the built-in battery 41 of the ultrasound probe 1 with the second charging power L2 smaller than the first charging power L1, the life of the built-in battery 41 can be maintained for a long time even in a case where the frequency of charging the built-in battery 41 is high as compared with the off state of the power supply of the ultrasound probe 1.

In a case where it is determined in step S4 that the ultrasound probe 1 is in the connection state with respect to the ultrasound apparatus main body 2, the processing proceeds to step S6. In step S6, the charging controller 21 charges the built-in battery 41 of the ultrasound probe 1 with third charging power L3, such as a charging voltage of 4.0 V, which is smaller than the second charging power L2 in step S5. In a case where the power supply of the ultrasound probe 1 is in the on state and the ultrasound probe 1 is connected to the ultrasound apparatus main body 2, for example, the ultrasound probe 1 is often used during the ultrasound examination of the subject. In this case, the built-in battery 41 may be frequently charged such that the remaining level of the built-in battery 41 during the ultrasound examination does not become insufficient, and the number of times the remaining level of the built-in battery 41 reaches the maximum capacity corresponding to the charging power used may increase. In addition, since the ultrasound probe 1 is used for the ultrasound examination, the power consumption is high as compared with a case where the ultrasound probe 1 is in the non-connection state with respect to the ultrasound apparatus main body 2, and the temperature of the built-in battery 41 is likely to increase. By charging the built-in battery 41 with the third charging power L3 smaller than the second charging power L2 in step S5, the life of the built-in battery 41 can be maintained for a long time even in a case where the frequency of charging the built-in battery 41 is high.

As described above, according to the ultrasound system according to the first embodiment of the present invention, the charging controller 21 charges the built-in battery 41 of the ultrasound probe 1 with charging power different depending on whether the power supply of the ultrasound probe 1 is in the on state or the off state, by using the power supplied from the outside of the ultrasound probe 1, so that the number of times of charging with the high charging power can be reduced while maintaining the life of the built-in battery 41 of the ultrasound probe 1, and the sufficient remaining level of the built-in battery 41 can be secured in the ultrasound examination. In addition, since the built-in battery 41 is charged with the charging power smaller than the first charging power L1 in the on state of the power supply of the ultrasound probe 1, the sufficient remaining level of the built-in battery 41 of the ultrasound probe 1 can be secured while the surface temperature of the ultrasound probe 1 is prevented from exceeding the upper limit temperature.

Although the built-in battery 41 of the ultrasound probe 1 is charged by wireless charging, the built-in battery 41 can also be charged by wired charging. A charging method of the built-in battery 41 is not particularly limited.

In addition, although the ultrasound probe 1 includes the image generation unit 13, the ultrasound apparatus main body 2 can also include the image generation unit 13.

In addition, although the ultrasound apparatus main body 2 is operated by the power supplied from the built-in battery 28, the ultrasound apparatus main body 2 can also be operated by being supplied with power from an external power supply by a so-called alternating current (AC) adapter or the like without including the built-in battery 28, for example.

Although the probe operation information acquisition unit 17 acquires whether the power supply of the ultrasound probe 1 is in the on state or the off state as the operation information of the ultrasound probe 1, the operation information of the ultrasound probe 1 can further include other information. The probe operation information acquisition unit 17 can further include, for example, wireless communication information related to the on state and the off state of the wireless communication circuit 14 of the ultrasound probe 1 as the operation information of the ultrasound probe 1. The wireless communication circuit 14 transmits and receives radio waves to and from the ultrasound apparatus main body 2 in the on state of the wireless communication circuit 14, and stops the transmission and reception of the radio waves to and from the ultrasound apparatus main body 2 in the off state of the wireless communication circuit 14. In the on state of the wireless communication circuit 14, the ultrasound probe 1 is often used for the ultrasound examination, and the ultrasound probe 1 may generate heat due to the transmission and reception of the radio waves by the wireless communication circuit 14.

In this case, in a case of controlling the charging power to the ultrasound probe 1 depending on the state of the power supply of the ultrasound probe 1, the charging controller 21 can control the charging power to the ultrasound probe 1 such that the charging power to the built-in battery 41 is decreased after the wireless communication circuit 14 is switched from the off state to the on state, and the charging power to the built-in battery 41 is increased after the wireless communication circuit 14 is switched from the on state to the off state. The charging controller 21 can charge the built-in battery 41 while preventing the temperature of the ultrasound probe 1 from exceeding the predetermined upper limit temperature by decreasing the charging power to the built-in battery 41 in the on state of the wireless communication circuit 14.

The probe operation information acquisition unit 17 can also include, for example, ultrasound transmission and reception information related to the on state and the off state of the transmission and reception circuit 12 in the operation information of the ultrasound probe 1. The transmission and reception circuit 12 transmits and receives the ultrasound waves to and from the inside of the subject from the transducer array 11 in the on state of the transmission and reception circuit 12, and stops the transmission and reception of the ultrasound waves to and from the inside of the subject from the transducer array 11 in the off state of the transmission and reception circuit 12. In the on state of the transmission and reception circuit 12, the ultrasound probe 1 is often used for the ultrasound examination, and the ultrasound probe 1 may generate heat due to the transmission and reception of the ultrasound waves from the transducer array 11.

In this case, in a case of controlling the charging power to the ultrasound probe 1 depending on the state of the power supply of the ultrasound probe 1, the charging controller 21 can control the charging power to the ultrasound probe 1 such that the charging power to the built-in battery 41 is decreased after the transmission and reception circuit 12 is switched from the off state to the on state, and the charging power to the built-in battery 41 is increased after the transmission and reception circuit 12 is switched from the on state to the off state. The charging controller 21 can charge the built-in battery 41 while preventing the temperature of the ultrasound probe 1 from exceeding the defined upper limit temperature by decreasing the charging power to the built-in battery 41 in the on state of the transmission and reception circuit 12.

The probe operation information acquisition unit 17 can also include, for example, the remaining level of the built-in battery 41 in the operation information of the ultrasound probe 1. In general, it is known that the battery is likely to deteriorate in a state in which the remaining level of the battery is near a fully charged state, that is, a state near 100% or in a state in which the remaining level of the battery is near an empty state, that is, a state near 0%. That is, it is known that it is preferable to maintain the remaining level of the battery at a medium level in order to suppress the deterioration of the battery.

In this case, in a case of controlling the charging power to the ultrasound probe 1 depending on the state of the power supply of the ultrasound probe 1, the charging controller 21 can control the charging power such that the charging power to the built-in battery 41 is larger as the remaining level of the built-in battery 41 is lower. The charging controller 21 can control the charging power to the built-in battery 41 by changing at least one of the charging voltage or the charging current to the built-in battery 41, for example. By controlling the charging power of the built-in battery 41 by taking into account the remaining level of the built-in battery 41, the charging controller 21 can prevent the remaining amount of the built-in battery 41 from being kept in an extremely small state or an extremely large state, and can prevent the deterioration of the built-in battery 41. The charging controller 21 can continuously change the charging power of the built-in battery 41 depending on the remaining level of the built-in battery 41, and can also change the charging power of the built-in battery 41 in stages.

The probe operation information acquisition unit 17 can also include, for example, plan information for performing the ultrasound examination in the operation information of the ultrasound probe 1. The plan information for performing the ultrasound examination includes, for example, information on a time slot in which the ultrasound examination is scheduled to be performed, such as a time slot from a specific first time to a second time in one day. The plan information for performing the ultrasound examination is input by the user via the input device 27 of the ultrasound apparatus main body 2, and is transmitted to the ultrasound probe 1 via the wireless communication circuit 22 of the ultrasound apparatus main body 2.

In this case, in a case of controlling the charging power to the ultrasound probe 1 depending on the state of the power supply of the ultrasound probe 1, the charging controller 21 can control the charging power such that the charging power to the built-in battery 41 is increased, by referring to plan information for performing the ultrasound examination in a case where there is a plan to perform the ultrasound examination within a predetermined time threshold, and the charging power to the built-in battery 41 is decreased immediately before the ultrasound examination. As a result, the built-in battery 41 can be charged to a sufficient remaining level before the start of the ultrasound examination, and the temperature of the ultrasound probe 1 can be sufficiently reduced at the start of the ultrasound examination.

In addition, in a case of controlling the charging power to the ultrasound probe 1 depending on the state of the power supply of the ultrasound probe 1, the charging controller 21 can charge the built-in battery 41 such that the remaining level of the built-in battery 41 is, for example, 80% or more, by referring to the plan information for performing the ultrasound examination in a case where there is a plan not to supply power for charging the built-in battery 41 from the charger 3 for a set time or longer. As a result, a sufficient remaining level of the built-in battery 41 of the ultrasound probe 1 can be secured until the start of the ultrasound examination.

In addition, in a case of controlling the charging power to the ultrasound probe 1 depending on the state of the power supply of the ultrasound probe 1, the charging controller 21 can charge the built-in battery 41 such that the remaining level of the built-in battery 41 is, for example, 30% to 70%, more preferably 40% to 60%, and more preferably 50% after completion of the last examination of the day. As a result, the remaining level of the built-in battery 41 can be prevented from being kept in an extremely small state or an extremely large state, and the deterioration of the built-in battery 41 can be prevented.

In addition, in a case of controlling the charging power to the ultrasound probe 1 depending on the state of the power supply of the ultrasound probe 1, the charging controller 21 can charge the built-in battery 41 such that the remaining level of the built-in battery 41 is, for example, 50% to 90% in an operation time slot of one day. As a result, the remaining level of the built-in battery 41 can be prevented from being kept in an extremely small state or an extremely large state, and the deterioration of the built-in battery 41 can be prevented.

The probe operation information acquisition unit 17 can also include, for example, information on an operation time of a facility that performs the ultrasound examination in the operation information of the ultrasound probe 1. In this case, in a case of controlling the charging power to the ultrasound probe 1 depending on the state of the power supply of the ultrasound probe 1, the charging controller 21 can increase the charging power to the built-in battery 41, by referring to the information on the operation time of the facility in a case where the facility starts operating within a defined time threshold. As a result, a sufficient remaining level of the built-in battery 41 of the ultrasound probe 1 can be secured until the start of the ultrasound examination.

The probe operation information acquisition unit 17 can also include, for example, a date and time set by the user in the operation information of the ultrasound probe 1. The user can set, for example, a date and time a certain time before the start of the scheduled ultrasound examination, or a start date and time of the scheduled ultrasound examination. Such a date and time is input by the user via the input device 27 of the ultrasound apparatus main body 2, and is transmitted from the wireless communication circuit 22 of the ultrasound apparatus main body 2 to the ultrasound probe 1.

In a case where the operation information of the ultrasound probe 1 includes the date and time set by the user, the charging controller 21 can increase the charging power to the built-in battery 41 or decrease the charging power to the built-in battery 41 at the date and time set by the user in a case of controlling the charging power to the ultrasound probe 1 depending on the state of the power supply of the ultrasound probe 1. The charging controller 21 can increase the charging power to the built-in battery 41 at the date and time a certain time before the start of the scheduled ultrasound examination, for example, so that a sufficient remaining level of the built-in battery 41 can be secured until the start of the ultrasound examination. In addition, the charging controller 21 can decrease the charging power to the built-in battery 41 at the start date and time of the scheduled ultrasound examination, for example, to suppress the increase in the surface temperature of the ultrasound probe 1.

### Second Embodiment

In general, in safety standards such as IEC 60601-2-37, which is an international standard related to individual requirements for basic safety and basic performance of a medical ultrasound diagnostic apparatus and a monitor device, it is defined that a surface temperature of the ultrasound probe 1 does not exceed an upper limit temperature such as 43° C. in order to prevent a burn of a subject and a user. Since the temperature of the ultrasound probe 1 is increased by transmitting and receiving the ultrasound waves via the transducer array 11 and charging the built-in battery 41 of the ultrasound probe 1, the built-in battery 41 may not be charged in a case where the temperature of the ultrasound probe 1 may exceed the predetermined upper limit temperature by the safety standard, for example, in a case where the built-in battery 41 is charged in a state where the power supply of the ultrasound probe 1 is turned on immediately after the ultrasound probe 1 is used. Therefore, the charging controller 21 can acquire the temperature of the ultrasound probe 1 as the operation information of the ultrasound probe 1, and can control the charging power to the built-in battery 41 based on the temperature of the ultrasound probe 1, for example.

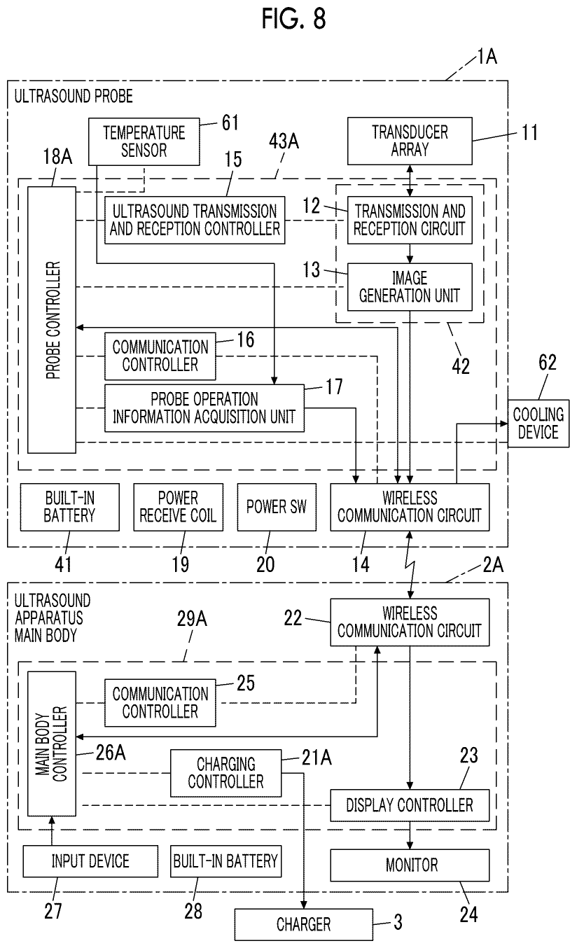

The ultrasound system according to a second embodiment includes an ultrasound probe 1A instead of the ultrasound probe 1 according to the first embodiment, and includes an ultrasound apparatus main body 2A instead of the ultrasound apparatus main body 2. FIG. 8 shows a configuration of the ultrasound probe 1A and the ultrasound apparatus main body 2A according to the second embodiment. The ultrasound probe 1A according to the second embodiment further includes a temperature sensor 61 (probe temperature sensor) and a cooling device 62, and includes a probe controller 18A instead of the probe controller 18 in the ultrasound probe 1 according to the first embodiment shown in FIG. 2. The temperature sensor 61 is connected to the wireless communication circuit 14 and the probe controller 18A. The cooling device 62 is connected to the probe controller 18A and the wireless communication circuit 14. A processor 43A for the ultrasound probe 1A is configured by the transmission and reception circuit 12, the image generation unit 13, the ultrasound transmission and reception controller 15, the communication controller 16, the probe operation information acquisition unit 17, and the probe controller 18A.

The ultrasound apparatus main body 2A according to the second embodiment includes a charging controller 21A instead of the charging controller 21, and includes a main body controller 26A instead of the main body controller 26 in the ultrasound apparatus main body 2 according to the first embodiment shown in FIG. 2. A processor 29A for the ultrasound apparatus main body 2A is configured by the charging controller 21A, the display controller 23, the communication controller 25, and the main body controller 26A.

The temperature sensor 61 of the ultrasound probe 1A detects the temperature of the ultrasound probe 1, for example, the temperature of the grip portion HB of the ultrasound probe 1. As the temperature sensor 61, for example, a resistive element such as a so-called thermistor, a so-called thermocouple, or a so-called radiation thermometer can be used. The information on the temperature of the ultrasound probe 1A detected by the temperature sensor 61 is transmitted to the ultrasound apparatus main body 2A via the wireless communication circuit 14.

The cooling device 62 is a device for cooling the ultrasound probe 1A. As the cooling device 62, for example, a cooling fan that blows air toward the ultrasound probe 1A to air-cool the ultrasound probe 1A can be used. As the cooling device 62, for example, a so-called Peltier element can also be used. The probe controller 18A can adjust the intensity of the cooling by adjusting the rotation speed of the cooling fan or the current amount in the Peltier element in the cooling device 62 based on the control information transmitted from the ultrasound apparatus main body 2A via the wireless communication circuit 14, or can turn on or off the operation of the cooling device 62.

The charging controller 21A of the ultrasound apparatus main body 2A can control the charging power from the charger 3 to the ultrasound probe 1A based on the temperature of the ultrasound probe 1A detected by the temperature sensor 61 instead of whether the power supply of the ultrasound probe 1A is in the on state or the off state. The charging controller 21A can decrease the charging power as the detected temperature of the ultrasound probe 1A is higher, and can increase the charging power as the detected temperature of the ultrasound probe 1A is lower, for example. As a more specific example, the charging controller 21A can charge the built-in battery 41 with first charging power L1 in a case where the temperature of the ultrasound probe 1A is equal to or lower than a first temperature threshold, can charge the built-in battery 41 with second charging power L2 lower than the first charging power L1 in a case where the temperature of the ultrasound probe 1A is higher than the first temperature threshold and equal to or lower than a second temperature threshold, and can charge the built-in battery 41 with third charging power L3 lower than the second charging power L2 in a case where the temperature of the ultrasound probe 1A is higher than the second temperature threshold. The second temperature threshold is lower than the first temperature threshold.

The charging controller 21A can further take into account the temperature of the ultrasound probe 1A detected by the temperature sensor 61 to control the charging power from the charger 3 to the ultrasound probe 1A in addition to whether the power supply of the ultrasound probe 1A is in the on state or the off state. The charging controller 21A can decrease the charging power as the temperature of the ultrasound probe 1A detected by the temperature sensor 61 is higher, and can increase the charging power as the temperature of the ultrasound probe 1A detected by the temperature sensor 61 is lower, for example.

Since the heat generation due to the charging of the ultrasound probe 1A can be suppressed by decreasing the charging power of the built-in battery 41, for example, even in a case where the temperature of the ultrasound probe 1A is increased by using the ultrasound probe 1A for the ultrasound examination of the subject, the built-in battery 41 can be charged while keeping the temperature of the ultrasound probe 1A lower than the predetermined upper limit temperature by the safety standard, so that the sufficient remaining level of the built-in battery 41 can be secured. In addition, since the heat generation during the charging of the built-in battery 41 can be suppressed by controlling the charging power of the built-in battery 41 by the charging controller 21A, for example, the user does not need to wait for a long time to lower the surface temperature of the ultrasound probe 1A in a case of using the ultrasound probe 1A for the ultrasound examination, and can immediately use the ultrasound probe 1A after the charging of the built-in battery 41.

The main body controller 26A of the ultrasound apparatus main body 2A can cool the ultrasound probe 1A by using the cooling device 62 depending on the temperature of the ultrasound probe 1A detected by the temperature sensor 61. The main body controller 26A can transmit control information to the cooling device 62 via the wireless communication circuit 22 such that the intensity of the cooling in the cooling device 62 is increased as the temperature of the ultrasound probe 1A is higher, and the intensity of the cooling in the cooling device 62 is decreased as the temperature of the ultrasound probe 1A is lower, for example. As a result, the increase in the surface temperature of the ultrasound probe 1A can be suppressed.

As described above, according to the ultrasound system according to the second embodiment, the charging controller 21A of the ultrasound apparatus main body 2A controls the charging power supplied from the charger 3 to the ultrasound probe 1A by taking into account the temperature of the ultrasound probe 1A detected by the temperature sensor 61, so that the sufficient remaining level of the built-in battery 41 of the ultrasound probe 1A can be secured while the surface temperature of the ultrasound probe 1A is prevented from exceeding the upper limit temperature.

The ultrasound probe 1A according to the second embodiment may not include the cooling device 62. In this case, the effect of keeping the surface temperature of the ultrasound probe 1A low is reduced, but the surface temperature of the ultrasound probe 1A can be sufficiently prevented from exceeding the upper limit temperature.

### Third Embodiment

In the second embodiment, although the ultrasound probe 1A includes the temperature sensor 61, the charger 3 that comes into contact with or is close to the ultrasound probe 1A during the charging can also include a temperature sensor 63 (charger temperature sensor).

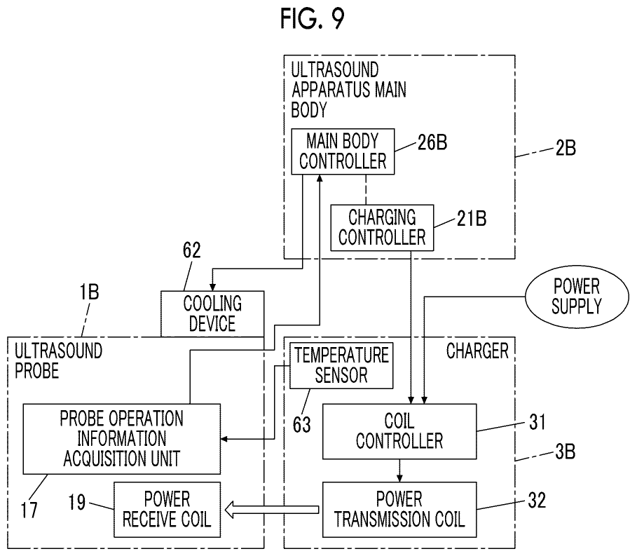

FIG. 9 shows a configuration of an ultrasound system according to a third embodiment. In FIG. 9, for convenience of description, a part of the internal configuration of an ultrasound probe 1B and an ultrasound apparatus main body 2B is omitted. The ultrasound probe 1B according to the third embodiment has a configuration in which the temperature sensor 61 is removed from the ultrasound probe 1A according to the second embodiment shown in FIG. 8. The ultrasound apparatus main body 2B according to the third embodiment includes a charging controller 21B instead of the charging controller 21A, and includes a main body controller 26B instead of the main body controller 26A in the ultrasound apparatus main body 2A according to the second embodiment shown in FIG. 8. The charger 3B according to the third embodiment further includes the temperature sensor 63 in the charger 3 according to the first embodiment and the second embodiment.

The temperature sensor 63 of the charger 3B detects the temperature of the charger 3B, for example, the temperature of a portion of the charger 3B that comes into contact with or is close to the ultrasound probe 1B during the charging of the ultrasound probe 1B, to indirectly detect the temperature of the ultrasound probe 1B. Examples of the portion that comes into contact with or is close to the ultrasound probe 1B include a vicinity of the power transmission coil 32. As the temperature sensor 63, for example, a resistive element such as a thermistor, a thermocouple, or a radiation thermometer can be used. The information on the temperature of the charger 3B detected by the temperature sensor 63 is transmitted to the ultrasound apparatus main body 2B.

The probe operation information acquisition unit 17 of the ultrasound probe 1B acquires information representing the temperature of the charger 3B detected by the temperature sensor 63 of the charger 3B as the operation information of the ultrasound probe 1B.

The charging controller 21B of the ultrasound apparatus main body 2B can control the charging power from the charger 3B to the ultrasound probe 1B based on the temperature of the charger 3B detected by the temperature sensor 63 instead of whether the power supply of the ultrasound probe 1B is in the on state or the off state. The charging controller 21B can decrease the charging power as the detected temperature of the charger 3B is higher, and can increase the charging power as the detected temperature of the charger 3B is lower, for example. As a more specific example, the charging controller 21B can charge the ultrasound probe 1B with first charging power L1 in a case where the temperature of the charger 3B is equal to or lower than a first temperature threshold, can charge the ultrasound probe 1B with second charging power L2 lower than the first charging power L1 in a case where the temperature of the charger 3B is higher than the first temperature threshold and equal to or lower than a second temperature threshold, and can charge the ultrasound probe 1B with third charging power L3 lower than the second charging power L2 in a case where the temperature of the charger 3B is higher than the second temperature threshold.

In addition, the charging controller 21B of the ultrasound apparatus main body 2B can further take into account the temperature of the charger 3B detected by the temperature sensor 63 to control the charging power from the charger 3B to the ultrasound probe 1B in addition to whether the power supply of the ultrasound probe 1B is in the on state or the off state. The charging controller 21B can decrease the charging power as the temperature of the charger 3B detected by the temperature sensor 63 is higher, and can increase the charging power as the temperature of the charger 3B detected by the temperature sensor 63 is lower, for example.

As described above, according to the ultrasound system according to the third embodiment, the charging controller 21B of the ultrasound apparatus main body 2B controls the charging power supplied from the charger 3B to the ultrasound probe 1B by taking into account the temperature of the charger 3B detected by the temperature sensor 63, so that the sufficient remaining level of the built-in battery 41 of the ultrasound probe 1B can be secured while the surface temperature of the ultrasound probe 1B is prevented from exceeding the upper limit temperature.

### Fourth Embodiment

The charging controller 21 of the ultrasound apparatus main body 2 can also control the charging power of the built-in battery 41 of the ultrasound probe 1 by taking into account the operation information of the ultrasound apparatus main body 2.

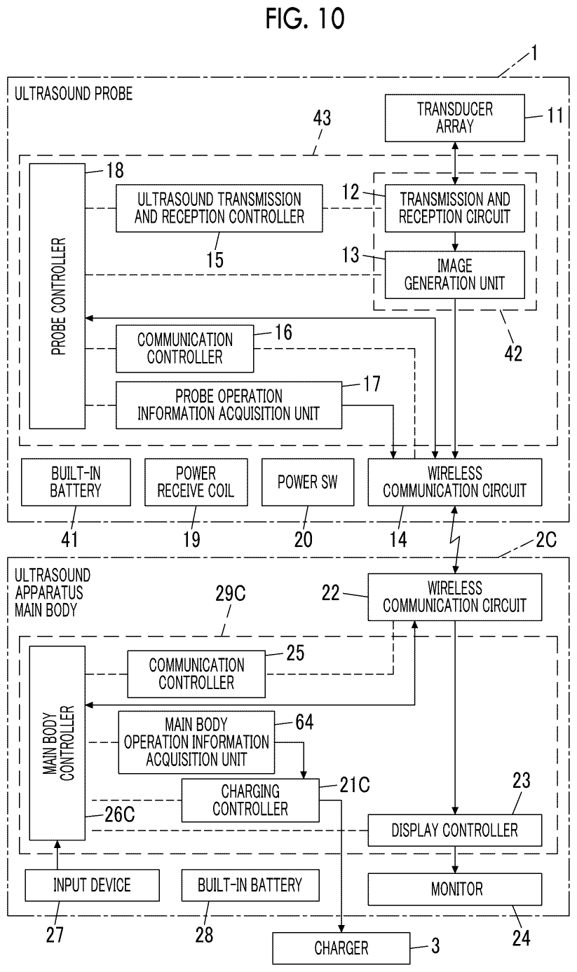

The ultrasound system according to a fourth embodiment includes an ultrasound apparatus main body 2C instead of the ultrasound apparatus main body 2 according to the first embodiment. FIG. 10 shows a configuration of the ultrasound apparatus main body 2C according to the fourth embodiment. The ultrasound apparatus main body 2C according to the fourth embodiment further includes a main body operation information acquisition unit 64, includes a charging controller 21C instead of the charging controller 21, and includes a main body controller 26C instead of the main body controller 26 in the ultrasound apparatus main body 2 according to the first embodiment shown in FIG. 2. The main body operation information acquisition unit 64 is connected to the charging controller 21 and the main body controller 26C. A processor 29C for the ultrasound apparatus main body 2C is configured by the charging controller 21C, the display controller 23, the communication controller 25, the main body controller 26C, and the main body operation information acquisition unit 64.

The main body operation information acquisition unit 64 acquires main body operation information in the ultrasound apparatus main body 2C. The main body operation information acquisition unit 64 can acquire, for example, power supply input information representing the on state of the power supply of the ultrasound apparatus main body 2C or wireless communication information representing the on state of the wireless communication circuit 22 of the ultrasound apparatus main body 2C as the main body operation information. In the on state of the power of the ultrasound apparatus main body 2C and the on state of the wireless communication circuit 22 of the ultrasound apparatus main body 2C, the ultrasound probe 1 is often used for the ultrasound examination, and the ultrasound probe 1 may generate heat due to the transmission and reception of the ultrasound waves from the transducer array 11.

In a case of controlling the charging power to the ultrasound probe 1 depending on the state of the power supply of the ultrasound probe 1, the charging controller 21C can control the charging power such that the charging power to the built-in battery 41 is decreased after receiving the power supply input information representing that the power supply of the ultrasound apparatus main body 2C is in the on state or the wireless communication information representing that the wireless communication circuit 22 of the ultrasound apparatus main body 2C is in the on state from the ultrasound apparatus main body 2C, and the charging power to the built-in battery 41 is increased after being unable to receive the power supply input information representing that the power supply of the ultrasound apparatus main body 2C is in the on state or the wireless communication information representing that the wireless communication circuit 22 of the ultrasound apparatus main body 2C is in the on state from the ultrasound apparatus main body 2C.

The main body operation information acquisition unit 64 can also acquire, for example, plan information for performing the ultrasound examination as the main body operation information. The plan information for performing the ultrasound examination is input by the user via the input device 27 of the ultrasound apparatus main body 2C, for example.

In this case, in a case of controlling the charging power to the ultrasound probe 1 depending on the state of the power supply of the ultrasound probe 1, the charging controller 21C can control the charging power such that the charging power to the built-in battery 41 is increased, by referring to the plan information for performing the ultrasound examination in a case where there is a plan to perform the ultrasound examination within a predetermined time threshold, and the charging power to the built-in battery 41 is decreased immediately before the ultrasound examination.

As described above, according to the ultrasound system according to the fourth embodiment, the charging controller 21C controls the charging power of the built-in battery 41 by taking into account the main body operation information acquired by the main body operation information acquisition unit 64, so that the sufficient remaining level of the built-in battery 41 of the ultrasound probe 1 can be secured while the surface temperature of the ultrasound probe 1 is prevented from exceeding the upper limit temperature.

The ultrasound apparatus main body 2C according to the fourth embodiment has a configuration in which the main body operation information acquisition unit 64 is added to the ultrasound apparatus main body 2 according to the first embodiment, but for example, the ultrasound apparatus main body 2C can also have a configuration in which the main body operation information acquisition unit 64 is added to the ultrasound apparatus main body 2A according to the second embodiment, and can also have a configuration in which the main body operation information acquisition unit 64 is added to the ultrasound apparatus main body 2B according to the third embodiment.

### EXPLANATION OF REFERENCES

1, 1A, 1B: ultrasound probe
2, 2A, 2B, 2C: ultrasound apparatus main body
3, 3B: charger
11: transducer array
12: transmission and reception circuit
13: image generation unit
14, 22: wireless communication circuit
15: ultrasound transmission and reception controller
16, 25: communication controller
17: probe operation information acquisition unit
18, 18A: probe controller
19: power receive coil
20: power switch (power SW)
23: display controller
24: monitor
26, 26A, 26C: main body controller
27: input device
28, 41: built-in battery
29, 29A, 29C, 43, 43A: processor
31: coil controller
32: power transmission coil
42: image acquisition unit
51: pulser
52: amplification section
53: AD conversion section
54: beam former
55: signal processing section
56: DSC
57: image processing section
61, 63: temperature sensor
62: cooling device
H: housing
HA: array accommodation portion
HB: grip portion

What is claimed is:

1. An ultrasound system in which an ultrasound probe including a transducer array and a built-in battery is wirelessly connected to an ultrasound apparatus main body including a processor,
wherein the processor is configured to charge the built-in battery of the ultrasound probe with charging power set according to operation information of the ultrasound probe by using power supplied from an outside of the ultrasound probe.

2. The ultrasound system according to claim 1,
wherein the operation information is information representing whether a power supply of the ultrasound probe is in an on state or an off state, and
the processor is configured to charge the built-in battery with charging power different depending on whether the power supply of the ultrasound probe is in the on state or the off state.

3. The ultrasound system according to claim 2,
wherein the processor is configured to charge the built-in battery with predetermined first charging power in the off state of the power supply, and to charge the built-in battery with charging power smaller than the first charging power in the on state of the power supply.

4. The ultrasound system according to claim 3,
wherein the processor is configured to charge the built-in battery with charging power different depending on whether the ultrasound probe is in a connection state of being wirelessly connected to the ultrasound apparatus main body or in a non-connection state of being not wirelessly connected to the ultrasound apparatus main body.

5. The ultrasound system according to claim 4,
wherein the processor is configured to charge the built-in battery with predetermined second charging power in the non-connection state, and to charge the built-in battery with third charging power smaller than the second charging power in the connection state.

6. The ultrasound system according to claim 1, further comprising:
a probe temperature sensor that detects a temperature of the ultrasound probe,
wherein the operation information is information representing the temperature detected by the probe temperature sensor, and
the processor is configured to control the charging power of the built-in battery based on the temperature.

7. The ultrasound system according to claim 2, further comprising:
a probe temperature sensor that detects a temperature of the ultrasound probe,
wherein the processor is configured to control the charging power of the built-in battery by taking into account the temperature of the ultrasound probe detected by the probe temperature sensor.

8. The ultrasound system according to claim 1, further comprising:
a charger that supplies power to the ultrasound probe; and
a charger temperature sensor that detects a temperature of the charger,
wherein the processor is configured to use information representing the temperature of the charger detected by the charger temperature sensor as the operation information,
to control the charging power of the built-in battery based on the temperature of the charger detected by the charger temperature sensor, and
to charge the built-in battery with the controlled charging power.

9. The ultrasound system according to claim 2, further comprising:
a charger that supplies power to the ultrasound probe; and
a charger temperature sensor that detects a temperature of the charger,
wherein the processor is configured to control the charging power of the built-in battery by taking into account the temperature of the charger detected by the charger temperature sensor, and
to charge the built-in battery with the controlled charging power by using the power supplied from the charger.

10. The ultrasound system according to claim 6, further comprising:
a cooling device that cools the ultrasound probe,
wherein the processor is configured to cool the ultrasound probe by using the cooling device according to the temperature of the ultrasound probe detected by the probe temperature sensor.

11. The ultrasound system according to claim 8, further comprising:
a cooling device that cools the ultrasound probe,
wherein the processor is configured to cool the ultrasound probe by using the cooling device according to the temperature of the charger detected by the charger temperature sensor.

12. The ultrasound system according to claim 1,
wherein the ultrasound probe includes a power receive coil, and
the processor is configured to charge the built-in battery by using the power wirelessly supplied from the outside of the ultrasound probe via the power receive coil.

13. The ultrasound system according to claim 1,
wherein the processor is configured to stop transmission and reception of ultrasound waves in the transducer array in a state where the built-in battery is being charged.

14. The ultrasound system according to claim 1,
wherein the processor is configured to maintain wireless connection between the ultrasound probe and the ultrasound apparatus main body in a state where the built-in battery is being charged.

15. The ultrasound system according to claim 1,
wherein the ultrasound probe includes a wireless communication circuit that performs wireless communication with the ultrasound apparatus main body, and
the processor is configured to control the charging power of the built-in battery by taking into account whether the wireless communication circuit is in an on state or an off state.

16. The ultrasound system according to claim 1,
wherein the ultrasound probe includes a transmission and reception circuit that performs transmission and reception of ultrasound waves using the transducer array, and
the processor is configured to control the charging power of the built-in battery by taking into account whether the transmission and reception circuit is in an on state or an off state.

17. The ultrasound system according to claim 1,
wherein the processor is configured to control the charging power of the built-in battery by taking into account a remaining level of the built-in battery.

18. The ultrasound system according to claim 1,
wherein the processor is configured to control the charging power of the built-in battery by taking into account a plan to perform an ultrasound examination.

19. The ultrasound system according to claim 1,
wherein the processor is configured to control the charging power of the built-in battery by taking into account an operation time of a facility that performs an ultrasound examination.

20. The ultrasound system according to claim 1,
wherein the processor is configured to control the charging power of the built-in battery by taking into account a date and time set by a user.

21. The ultrasound system according to claim 1,
wherein the processor is configured to control the charging power of the built-in battery by taking into account operation information of the ultrasound apparatus main body.

22. A control method of an ultrasound system in which an ultrasound probe including a transducer array and a built-in battery is wirelessly connected to an ultrasound apparatus main body, the control method comprising:
charging the built-in battery of the ultrasound probe with charging power set according to operation information of the ultrasound probe by using power supplied from an outside of the ultrasound probe.
